(19) United States
(12) Patent Application Publication
(10) Pub. No.: US 2014/0156549 A1
(43) Pub. Date: Jun. 5, 2014

(54) METHOD FOR ESTIMATING CARBON SEQUESTRATION IN GRASSLANDS

(71) Applicant: Mark Ritchie, Syracuse, NY (US)

(72) Inventor: Mark Ritchie, Syracuse, NY (US)

(73) Assignee: SYRACUSE UNIVERSITY, Syracuse, NY (US)

(21) Appl. No.: 14/084,567

(22) Filed: Nov. 19, 2013

Related U.S. Application Data
(60) Provisional application No. 61/727,791, filed on Nov. 19, 2012.

(51) Int. Cl. G01N 33/24; G06Q 30/00

(52) U.S. Cl. CPC G01N 33/24; G06Q 30/018
USPC 705/317; 702/189

(57) ABSTRACT

A method of estimating the soil organic carbon (SOC) stocks for a grassland based on the plant-derived SOC plus the dung-derived SOC minus the carbon lost through microbial maintenance respiration. The plant-derived SOC, dung-derived SOC, and microbial maintenance respiration are estimated by considering the effect of one or more of the lignin and cellulose content of the plant material, the estimated annual aboveground and belowground plant production, the grazing intensity, the number of fires per year, the mean annual rainfall, the belowground net primary production, and the soil texture. The method thus provides an allowance of plants to compensate for grazing without losing leaf area, and the diversion of carbon through grazing animals and into soil through the deposit of dung.

### CROSS-REFERENCE TO RELATED APPLICATIONS

This application claims priority to U.S. Provisional Application No. 61/727,791, filed on Nov. 19, 2012.

### STATEMENT REGARDING FEDERALLY SPONSORED RESEARCH

This invention was made with government support under DEB 0842230 and DEB 0543398 awarded by the National Science Foundation (NSF). The government has certain rights in the invention.

### BACKGROUND OF THE INVENTION

1. Field of the Invention

The present invention relates to carbon sequestration modeling and, more particularly, to an accurate method for estimating carbon sequestration in tropical grasslands.

2. Description of the Related Art

Soil organic carbon (SOC) in grasslands and savannas represents one of the largest reservoirs of carbon on earth and thus one of the most important potential sinks of carbon dioxide in the effort to mitigate climate change. Understanding the dynamics of SOC is thus of paramount importance for scientists and policy-makers. A major question is what management practices in grasslands, in the form of grazing, fire, fertilization, re-vegetation and restoration, etc., can lead to net sequestration of carbon. Because SOC changes often require years to reach a level detectable against large SOC stocks, models represent key tools for assessing the consequences of management.

A wealth of field studies in North America, Europe, and increasingly in Asia, have supported modeling of SOC for temperate grasslands. This effort has culminated in large, complex, and mostly successful models such as CENTURY and RothC that require entry of or draw from empirical databases to make default selections of a large number of parameters. Some of the many factors incorporated into these models include precipitation, soil texture, pH, exchangeable bases, temperature, grazing, fire frequency, soil nutrients, plant tissue nutrients, etc. The models typically track two or more SOC pools that differ in their turnover of carbon, and consider changes in parameter values over relatively fine time scales (days, weeks, or months). These models generally predict well changes in SOC related to production and decomposition, but can they can be insensitive to changes related to the management of grasslands, namely fire and grazing.

Tropical grasslands and savannas occupy nearly 10% of the earth's land surface and have measured SOC stocks from 10-120 metric tons(mt)/ha. However the dynamics of SOC in response to changes in fire, grazing, and other management in tropical systems are virtually unstudied. Tropical grasslands feature several characteristics that may cause them to function differently than temperate systems, and exhibit different SOC dynamics. First, they are almost entirely dominated by warm-season (C4) grasses that invest heavily in rhizomes and other storage organs that allow them to respond quickly to rainfall and to defoliation. In particular, compensatory responses to grazing can involve a reduction in allocation to stem and an increase in specific leaf area, which might sustain ecosystem photosynthetic capacity despite removal of considerable production. Second, these grasses contain higher levels of lignin and cellulose, which are generally recalcitrant to decomposition. Thirdly, high seasonal rainfall can lead to intense periods of production followed by drier periods during which standing biomass is highly vulnerable to and frequently experiences fire. Finally, benign temperatures allow for the prevalence of macro-decomposers, such as termites and dung beetles, which often rapidly incorporate senesced plant material and herbivore dung into soil. These and other features suggest that carbon fixation may be relatively insensitive to grazing and fire, that fixed carbon may be less decomposable, and that combustion and incorporation of dung into soil organic matter may represent important and fates of fixed carbon not prevalent in many temperate systems.

Although the most successful temperate SOC models have not been tested in tropical ecosystems, the large numbers of parameter inputs they require to make good predictions are virtually unavailable from the less intensively studied tropics. For example, CENTURY will select parameters by default from internal databases, but these data may not represent tropical conditions or functional relationships as discussed in Paustian, K., W. J. Parton, and J. Persson, Modeling soil organic-matter in inorganic-amended and nitrogen-fertilized long-term plots, Soil Science Society of America Journal 56:476-488 (1992), hereby incorporated by reference and referred to herein as Paustian. Thus, a simpler modeling approach with fewer inputs might be necessary at this point to explore SOC dynamics to even a first approximation for tropical habitats.

For example, one study found that a relatively simple model of nitrogen dynamics, with fine time scales of resolution but consideration of relatively few pools and fluxes, described well the impacts of grazing animals and fire on soil N in the Serengeti, see Holdo, R. M., R. D. Holt, M. B. Coughenour, and M. E. Ritchie, Plant productivity and soil nitrogen as a function of grazing, migration and fire in an African savanna, Journal of Ecology 95:115-128 (2007), hereby incorporated by reference and referred to herein as Holdo. Empirical studies of SOC dynamics in the tropics suggest that relatively few factors may explain the majority of variation in SOC and key processes that affect it, like soil microbial respiration and termite decomposition of plant litter, see Wilsey, B. J., G. Parent, N. T. Roulet, T. R. Moore, and C. Potvin, Tropical pasture carbon cycling: relationships between C source/sink strength, aboveground biomass, and grazing, Ecology Letters 5:367-376 (2002) hereby incorporated by reference and referred to herein as Wilsey. Consequently, there is a need for a process that estimates SOC using a highly simplified model of SOC dynamics to explain the considerable variation in SOC stocks in tropical grasslands.

### BRIEF SUMMARY OF THE INVENTION

The present invention provides a method for estimating the sequestration of carbon in grazed grasslands. The present invention considers the major pathways of the fate of fixed carbon (e.g., incorporation in biomass, combustion, consumption, dung deposition, respiration) and the mechanisms that prevalent in and unique to tropical systems, such as compensatory responses to defoliation. More specifically, the method of the present invention is based on a simplified model of SOC dynamics for tropical grassland that operates as a function of five input variables: mean annual rainfall, grazing intensity, fire frequency, aboveground percent cellulose plus lignin, and soil texture (percent sand), along with several key grassland parameters.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic showing the major fates of carbon in tropical grassland;

FIGS. 2A through 2F are graphs illustrating the key parameter relationships underlying the method of the present invention;

FIG. 3A trough 3D are graphs illustrating the predictions of the present invention as a function of the key input variables of grazing intensity and fire frequency for different combinations of low (RAIN=450) and high (RAIN=800) rainfall and low (SAND %=25, Fine-Textured Soils) and high (SAND %=65, Coarse-Textured) soil sand content, where the lignin and cellulose content (LIGCELL) was set at 0.3;

FIG. 4 is a graph comparing the estimates provided by the method of the present invention with actual measurements taken at eight test locations.

### DETAILED DESCRIPTION OF THE INVENTION

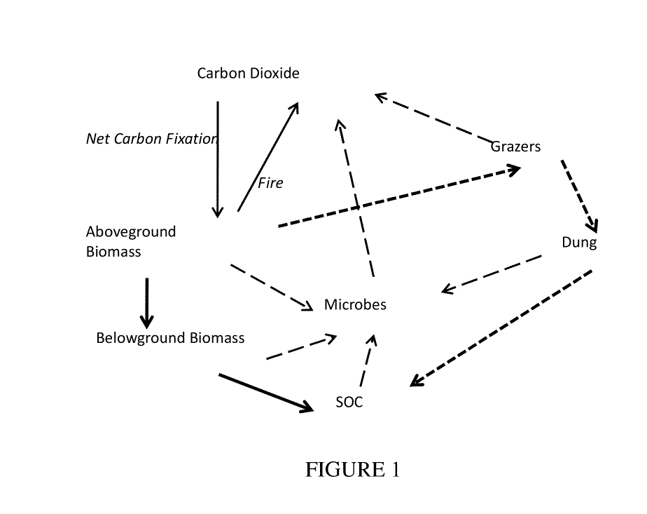

Referring now to the drawings, wherein like reference numerals refer to like parts throughout, there is seen in FIG. 1 a model of the major fates of carbon in tropical grassland considered by the present invention as part of a practical soil carbon dynamic model to allow for the estimation of soil organic carbon over time. Net fixed carbon becomes resident soil organic carbon (SOC) through two major pathways: plant-derived SOC not consumed by microbes (in soil or the guts of macro-decomposers like termites) (heavy solid arrows), and dung-derived SOC (heavy short-dashed arrows) not assimilated by grazers and gut or soil microbes. All other carbon is combusted through fire (black arrow), or respired by grazers and microbes in soil or the guts of macro-decomposers (long dashed arrows).

The method of the present invention is based on a number of observations about the SOC dynamics of tropical grasslands. First, tropical grasses exhibit compensatory responses to defoliation that maintain similar leaf area, and thus photosynthetic capacity, across a broad range of grazing intensities. Second, the largest carbon inputs to the soil organic matter pool occur through decomposition of aboveground and belowground biomass and through incorporation of herbivore dung into soil. Third, the major losses of SOC derive from combustion (fire), herbivore respiration, and soil microbial respiration. Finally, all plant tissue, other than lignin and cellulose, is assumed to be respired through by herbivores and by microbes in soil or the guts of macro-decomposers.

Using these observations, the present invention provides a simplified method for analyzing SOC dynamics for tropical grassland as a function of five input variables, i.e., mean annual rainfall, grazing intensity, fire frequency, lignin and cellulose content, and soil texture (percent sand), as well as a few standard parameters. Using these input variables and parameters, the method of the present invention can be used to estimate the SOC for a particular location and to provide an estimate of the change in SOC over time.

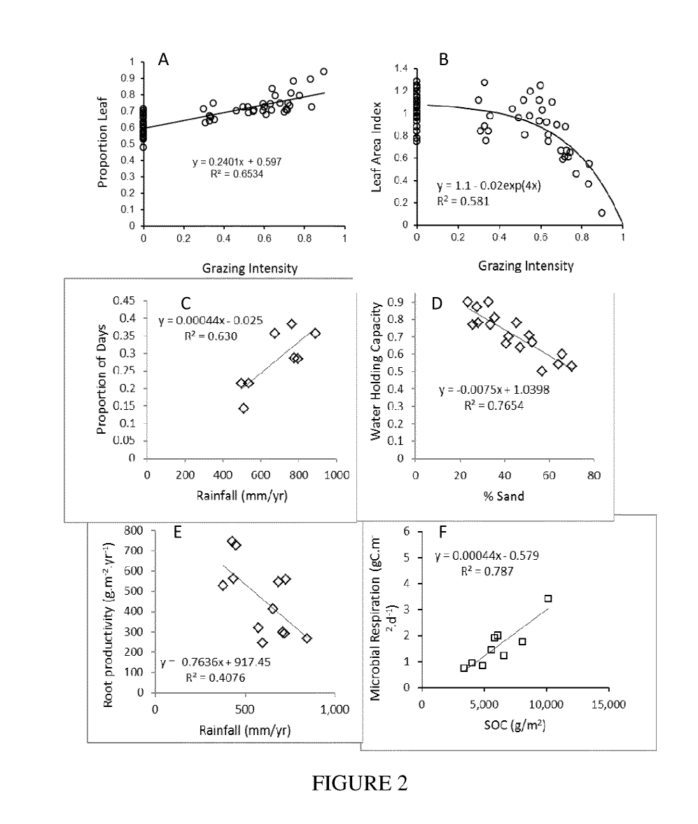

Referring to FIG. 2A, the method of the present invention considers the proportion of leaves (P_{L}) in a given location. As seen in FIG. 2B, a leaf area index (LAI, cm²/cm²) of grazed and ungrazed plants as a function of grazing intensity (GI) may also also considered. As seen in FIG. 2C, the number of moist soil days (WETDAYS) as a function of annual rainfall (RAIN) is also considered. Other parameters taken into consideration by the method of the present invention are known in the art. For example, as seen in FIG. 2C, the decline in water-holding capacity of soils with increasing sand content, see Ruess, R. W. and S. W. Seagle, Landscape patterns in soil microbial processes in the Serengeti National Park. Ecology 75:892-904 (1994), hereby incorporated by reference and referred to herein as Ruess, is one parameter considered in the present invention. In addition, as seen in FIG. 2D, the annual belowground production as a function of rainfall, as described in McNaughton, S. J., F. F. Banyikwa, and M. M. McNaughton, Root biomass and productivity in a grazing ecosystem: the Serengeti. Ecology 79:582-592 (1998) hereby incorporated by reference and referred to herein as McNaughton (1998), is taken into consideration. Finally, as seen in FIG. 2E, the microbial maximum respiration rates as a function of soil carbon stocks ((SOC) as discussed in Ruess), were also used in the method of the present invention.

The first step in the method of the present invention is to calculate the maximum aboveground net primary productivity (ANPPₜ^{max}) using annual rainfall in millimeters (RAIN). The equation for calculating the maximum aboveground net primary productivity (ANPPₜ^{max}) according to McNaughton, S. J., Ecology of a grazing ecosystem: The Serengeti. Ecological Monographs 55 259-294 (1985) hereby incorporated by reference and referred to herein as McNaughton (1985), weighted by the water holding capacity (WHC) that, according to Ruess, is negatively related to the sand content (SAND %) of the soil:

ANPPₜ^{max}=(0.84*RAIN−27.5)*(1.315−0.007*SAND %)

Using the aboveground net primary productivity (ANPPₜ^{max}) determined above, it is then possible to calculate the estimated aboveground net primary productivity (ANPPₜ^{est}) based on the leaf area index, LAI as follows:

ANPPₜ^{est}=LAI*ANPPₜ^{max}

The next step in the process is to determine a leaf area index (LAI). The leaf area index (LAI) is based upon the proportion of leaves (P_{L}), which is in turn a function of the grazing intensity (GI). Grazing intensity (GI) is the second variable input of the present invention and comprises the fractional difference between standing aboveground biomass (AGB) outside a grazing enclosure verses inside a grazing enclosure. Grazing intensity (GI) is not a direct measure of the fraction of annual production consumed by grazing animals, but instead reflects the degree to which grazing reduces standing fuel for fires and provides a lower bound on an estimate of the fraction of aboveground production that is converted into dung. Grazing intensity (GI) may be calculated, as discussed in McNaughton (1985), from the difference in aboveground biomass under ungrazed conditions (ABG_{ug}) and aboveground biomass under grazed conditions (ABG_{g}):

GI=1−AGB_{g}/ABG_{g}

The proportion of leaves (P_{L}) may be calculated from the grazing intensity (GI) according to the following:

P_{L}=0.6+0.24*GI

Using the proportion of leaves (P_{L}), as well as the maximum aboveground net primary productivity (ANPPₜ^{max}) and annual rainfall (RAIN), the leaf area index (LAI) may be calculated as follows:

LAI=(P_{I}/0.6)−0.015(ANPPₜ^{max}/RAIN)exp(4.6*GI)

Once the leaf area index (LAI) is determined, an estimated aboveground net primary productivity (ANPPₜ^{est}) may be calculated as follows:

ANPPₜ^{est}=ANPPₜ^{max}*LAI

Thus, using the maximum amount of productivity possible and factoring in the proportion of leaves and the leaf area index, the method of the present invention can estimate the actual amount of productivity of the grassland at issue.

In addition to an estimated aboveground net primary productivity (ANPPₜ^{est}), the method of the present invention considers the estimated belowground net primary production (BNPPₜ^{est}) for the given location. According to McNaughton, (1998) and McNaugton, (1985), the estimated belowground net primary production (BNPPₜ^{est}) for a grassland may be calculated based on the annual rainfall using the following formula:

BNPPₜ^{est}=917.4 −0.763*RAIN

Once the estimated aboveground net primary productivity (ANPPt^{est}) and estimated belowground net primary production (BNPPₜ^{est}) are determined, the plant derived SOC (PDSOC) may be calculated from the fire frequency (FIRE) and the lignin and cellulose content (LIGCELL). The fire frequency (FIRE) represents the number of fires every ten years or, alternatively, the fraction of landscape burned each year on average. The fire frequency may be measured based on the burned and unburned areas during the late dry season using satellite images, such as MODIS' Burned Area Product, or similar validated technique. An acceptable method for measuring fire frequency based on satellite image is explained in Dempewolf et al., I.E.E.E. Geoscience and Remote Sensing Letters 4(2): 312-316 (2007), hereby incorporated by reference.

The lignin and cellulose content (LIGCELL) is determined based on the lignin and cellulose fraction of aboveground tissue. If the lignin and cellulose content of the aboveground plant tissue is not known, it may be measured by sequential digestion of tissue material. For example, all plant material from a given plot, such as 15 by 15 centimeter square, may be clipped and dried. The clipped and dried material may then be digested in an acid detergent to remove the non-lignin, non-cellulose portions, digested in concentrated sulfuric acid, to remove the cellulose, and then subject to ashing at 400 degrees C. for 24 hours to remove lignin, leaving behind only minerals.

The plant derived SOC (PDSOC) may then be calculated from fire frequency (FIRE) and the lignin and cellulose content (LIGCELL) as follows, where 0.45 represents the carbon content of plant material, as discussed in Tao, G. C., T. A. Lestander, P. Geladi, and S. J. Xiong, Biomass properties in association with plant species and assortments I: A synthesis based on literature data of energy properties, Renewable & Sustainable Energy Reviews 16:3481-3506 (2012), hereby incorporated by reference in its entirety, as follows:

PDSOCₜ=LIGCELL*0.45*[ANPPₜ^{est}*(1−GI)*(1−FIRE)+BNPPₜ^{est}]

In this manner, the method of the present invention accounts for carbon losses as a result of the digested portion of the plant material as a well as losses attributable to fire.

In addition to the plant derived SOC (PDSOC), the present invention further considers the amount of dung derived SOC (DDSOC) that is placed in the soil based on the lignin and cellulose content (LIGCELL) of the plant material, the carbon content of plant material, the grazing intensity (GI) calculated above, and the aboveground net primary productivity (ANPPₜ^{est}) calculated above, as follows:

DDSOCₜ=LIGCELL*0.45*GI*ANPPₜ^{est}

In calculating total SOC, it is also necessary to account for carbon losses associated with maintenance respiration (MRESPₜ). Maintenance respiration (MRESPₜ) is a function of the number of wet days (WETDAYS) which is, in turn, determined based on the average annual rain fall as follows as derived from additional data collected by the inventor:

WETDAYS=(0.00044*RAIN−0.025)*240

Once the wet days are calculated, the microbial maintenance respiration (MRESPₜ) may be calculated based on the number of wet days (WETDAYS) and the soil carbon stocks (SOCₜ), adjusted for soil sand content (SAND %) using the formula derived with data from Ruess and Seagle (1994), hereby incorporated by reference:

MRESPₜ=WETDAYS*(0.7+0.3*SAND %/100)*(0.00044*SOCₜ−0.579)

Finally, the change in sequestered carbon (ΔSOCt) can be estimated by adding the plant derived SOC (PDSOCₜ) to the dung derived SOC (DDSOCₜ) and then subtracting the carbon lost through microbial maintenance respiration (MRESPₜ) as follows:

ΔSOCt=PDSOCt+DDSOCt−MRESPt

By setting ΔSOCt=0, the above equation can be solved for the SOCt term in MRESPt to yield an equilibrium SOC_{eq}.

SOC_{eq}=[PDSOCt+DDSOCt+WETDAYS*(0.579)*(0.7+0.3*SAND %/100)]/[(0.00044*WETDAYS*(0.7+0.3*SAND %/100)]

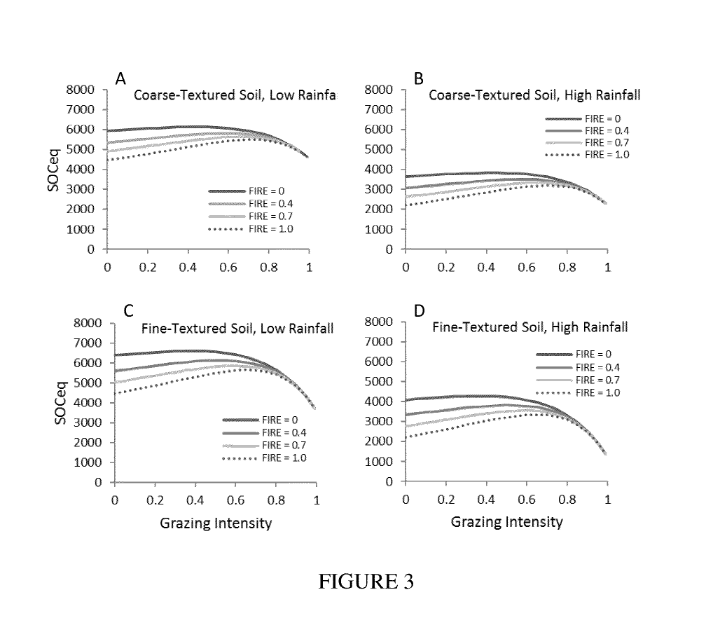

Referring to FIG. 3A through 3D, the estimates provided by the method of the present invention are a function of the key input variables of grazing intensity and fire frequency for different combinations (of low (RAIN=450) and high (RAIN=800) rainfall and low (SAND %=25, Fine-Textured Soils) and high (SAND %=65, Coarse-Textured) soil sand content, where the lignin and cellulose content (LIGCELL) was set at 0.3.

The accuracy of the estimated SOC provided by the method of the present invention with respect to eight discrete grassland locations was evaluated against actual samples collected at those sites. The eight sites varied widely in rainfall, grazing intensity, fire frequency, and soil texture. The method of the present invention fit the observed data extremely well (R²=0.95), establishing that the present invention captures important pathways of carbon transfer and, in particular, the importance of plant compensation to grazing and the importance of dung inputs to SOC.

Table 3 below sets forth the actual measured characteristics for the eight grazed grassland sited used to evaluate the accuracy of the method of the present invention. At the sites, the grazing intensity, soil sand content, and aboveground tissue lignin and cellulose were measured, and the mean annual rainfall and fire frequency over the previous nine years were known.

TABLE 3
Mean (±SE) characteristics of the eight study sites in the grazing experiment.

| | Grazing | Ungrazed | Lignin + | Rainfall | | | |
|---|---|---|---|---|---|---|---|
| | Intensity | Biomass | Cellulose | 1999-2008 | | Soil N | Soil C |
| | (%) | (g/m², | (%) | (mm/y, | Fires, | (%, | (%, |
| Site | (N = 3) | N = 3) | (N = 3) | N = 9) | 2000-2008 | N = 6) | N = 6) |
| Balanites | 32 ± 14 | 847 ± 133 | 34.1 ± 2.3 | 721 ± 86 | 4 | 0.19 ± 0.05 | 1.84 ± 0.13 |
| Barafu | 65 ± 4 | 605 ± 66 | 34.5 ± 1.9 | 472 ± 31 | 2 | 0.26 ± 0.07 | 3.14 ± 0.06 |
| Klein's | 56 ± 9 | 691 ± 71 | 36.3 ± 2.4 | 771 ± 61 | 5 | 0.22 ± 0.03 | 1.77 ± 0.09 |
| Camp | | | | | | | |
| West | | | | | | | |
| Kemaris | 66 ± 3 | 643 ± 27 | 31.6 ± 3.1 | 832 ± 87 | 4 | 0.25 ± 0.06 | 2.67 ± 0.08 |
| che Hills | | | | | | | |
| Kuka | 49 ± 4 | 623 ± 20 | 37.7 ± 2.4 | 784 ± 41 | 5 | 0.13 ± 0.04 | 2.13 ± 0.03 |
| Hills | | | | | | | |
| Musabi | 28 ± 13 | 827 ± 251 | 37.6 ± 1.9 | 885 ± 63 | 7 | 0.14 ± 0.07 | 2.20 ± 0.21 |
| Plains | | | | | | | |
| Soit | 54 ± 13 | 292 ± 53 | 35.6 ± 2.1 | 499 ± 39 | 4 | 0.11 ± 0.02 | 1.91 ± 0.14 |
| Olowotonyi | | | | | | | |
| Tagora | 69 ± 12 | 744 ± 27 | 31.8 ± 2.8 | 654 ± 87 | 5 | 0.15 ± 0.06 | 1.85 ± 0.07 |
| Plains | | | | | | | |

| | | | | | Bulk |
|---|---|---|---|---|---|
| | | Sand | Silt | Clay | Density |
| | Soil P | (%, | (%, | (%, N = | (g/cm³, |
| Site | (%, N = 6) | N = 6) | N = 6) | 6) | N = 6) |
| Balanites | 0.0325 ± 0.0030 | 51.0 ± 2.5 | 38.3 ± 4.2 | 10.7 ± 2.2 | 1.31 ± 0.14 |
| Barafu | 0.1132 ± 0.0038 | 27.6 ± 2.2 | 60.6 ± 4.1 | 11.7 ± 2.0 | 0.85 ± 0.12 |
| Klein's | 0.0059 ± 0.0008 | 40.6 ± 3.1 | 35.5 ± 3.3 | 23.9 ± 2.4 | 1.07 ± 0.17 |
| Camp | | | | | |
| West | | | | | |
| Kemaris | 0.0500 ± 0.0086 | 35.5 ± 2.7 | 52.0 ± 1.8 | 12.5 ± 2.8 | 0.96 ± 0.04 |
| che Hills | | | | | |
| Kuka | 0.0075 ± 0.0005 | 45.4 ± 1.2 | 46.2 ± 2.8 | 8.40 ± 1.3 | 1.15 ± 0.12 |
| Hills | | | | | |
| Musabi | 0.0692 ± 0.0063 | 32.9 ± 4.6 | 31.2 ± 3.6 | 35.9 ± 2.9 | 0.90 ± 0.10 |
| Plains | | | | | |
| Soit | 0.1240 ± 0.0052 | 32.1 ± 4.1 | 55.4 ± 4.1 | 12.5 ± 2.4 | 0.84 ± 0.08 |
| Olowotonyi | | | | | |
| Tagora | 0.0612 ± 0.0030 | 65.8 ± 3.7 | 28.0 ± 1.5 | 6.21 ± 1.3 | 1.22 ± 0.17 |
| Plains | | | | | |

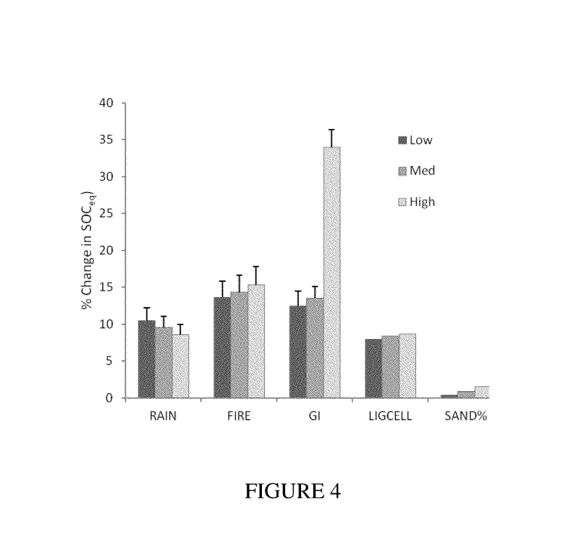

As seen in FIG. 4, the predicted equilibrium soil organic carbon stocks (g/m² to 40 cm depth) SOC_{eq} were obtained using the present invention by solving for SOC when ΔSOCt=0. Calculations of SOC_{eq} were accurate when compared to mean observed soil carbon stocks (N=8), thereby demonstrating the accuracy of the present invention. The slope 1.088±0.056 (SE) is not significantly different from 1 (P=0) and the intercept −192.8±567 (SE) is not significantly different from zero (P=0.91), indicating that the model is unbiased. It should be recognized by those of skill in the art that the present invention may be implemented via a computer spreadsheet or by a dedicated computer program or application that is programmed to accept entry of the information and perform the appropriate calculations.

The method of the present invention may be used to estimate soil organic carbon stocks for the purposes of obtaining certification for a particular carbon credit project and for calculating the appropriate number of carbon credits generated by the project. Using the present invention, a carbon project developer that wants to start a carbon credit projects in a tropical grassland where cattle grazing or fire occurs can perform accurate modeling of soil carbon changes in order to claim carbon credits on a periodic basis, thereby avoiding the need to wait years for the soil carbon changes to be detectable.

To be used in a carbon credit project, the present invention must be validated for the project area by showing its ability to predict initial carbon stocks as a consequence of past management actions and conditions, such as rainfall, plant species composition, grazing intensity, fire history, etc., in different subareas (strata) within the project area that differ strongly in past conditions or in management activities. The project area-validated model is used first to back-cast soil carbon dynamics to assess the maximum SOC that likely occurred in the previous 10 years as the baseline SOC, as required by the Verified Carbon Standard as an uncertainty deduction for activity-based projects. The same model is then used to calculate an expected future equilibrium SOC under proposed project activities, the time in years to reach this equilibrium, and the average annual increment in SOC sequestration expected under the proposed project activities.

Typically, the user would define a project area and identify or measure the key input parameters of the present invention, namely, mean annual rainfall (RAIN), mean grazing intensity that has been in effect for the previous 10-30 years (GI), aboveground plant lignin and cellulose content (LIGCELL), fire frequency (FIRE), and soil texture (SAND %) that apply to that area. The user would then calculate, with the present invention, the equilibrium SOC under these historical conditions SOC_{eq} and then calculate SOC that occurred 10 years earlier as the maximum SOC that occurred in the past 10 years as a conservative starting point for the carbon project, SOC₀. Then, the SOC at equilibrium under a proposed carbon project activities in the project area can be calculated (SOC_{act}). These two calculations from the present invention would then be used to calculate the average annual change in soil carbon that would result from the project activities, which is then used to determine the number of carbon credits that can be claimed from the project.

What is claimed is:

1. A method of determining a change in sequestered carbon in a predetermined area of a grassland, comprising the steps of:
calculating a maximum aboveground net primary productivity based on an annual rainfall amount;
determining a leaf area index based on a proportion of leaves and a grazing intensity;
calculating an estimated aboveground net primary productivity based on said leaf area index and said maximum aboveground net primary productivity calculating an estimated belowground net primary production;
determining a plant derived soil organic carbon from a fire frequency, a measurement of the lignin and cellulose content of the aboveground plant tissue, and said estimated aboveground net primary productivity;
calculating an amount of dung derived soil organic carbon based on said measurement of the lignin and cellulose content of the aboveground plant tissue, said grazing intensity, and said aboveground net primary productivity;
calculating a carbon loss associated with maintenance respiration; and
determining a change in sequestered carbon by adding said plant derived soil organic carbon to said dung derived soil organic carbon and subtracting said carbon loss associated with maintenance respiration.

2. The method of claim 1, wherein the maximum aboveground net primary productivity based on annual rainfall is weighted by a water holding capacity calculated from a sand content in the predetermined area.

3. The method of claim 1, wherein said grazing intensity comprises the difference in an aboveground biomass under ungrazed conditions and an aboveground biomass under grazed conditions.

4. The method of claim 1, wherein said fire frequency comprises a measurement of the number of fires in said predetermined location every ten years.

5. The method of claim 1, wherein said fire frequency comprises a measurement of the fraction of landscape burned each year on average.

6. The method of claim 1, wherein said lignin and cellulose content of the aboveground plant tissue is measured by sequential digestion of tissue material in a predetermined plot with said predetermined location.

7. A system for determining a change in sequestered carbon in a predetermined area of a grassland, comprising:
means for calculating a maximum aboveground net primary productivity based on an annual rainfall amount;
means for determining a leaf area index based on a proportion of leaves and a grazing intensity;
means for calculating an estimated aboveground net primary productivity based on said leaf area index and said maximum aboveground net primary productivity
means for calculating an estimated belowground net primary production;
means for determining a plant derived soil organic carbon from a fire frequency, a measurement of the lignin and cellulose content of the aboveground plant tissue, and said estimated aboveground net primary productivity;
means for calculating an amount of dung derived soil organic carbon based on said measurement of the lignin and cellulose content of the aboveground plant tissue, said grazing intensity, and said aboveground net primary productivity;
means for calculating a carbon loss associated with maintenance respiration; and
means for determining a change in sequestered carbon by adding said plant derived soil organic carbon to said dung derived soil organic carbon and subtracting said carbon loss associated with maintenance respiration.

8. The system of claim 1, wherein the maximum aboveground net primary productivity based on annual rainfall is weighted by a water holding capacity calculated from a sand content in the predetermined area.

9. The system of claim 1, wherein said grazing intensity comprises the difference in an aboveground biomass under ungrazed conditions and an aboveground biomass under grazed conditions.

10. The system of claim 1, wherein said fire frequency comprises a measurement of the number of fires in said predetermined location every ten years.

11. The system of claim 1, wherein said fire frequency comprises a measurement of the fraction of landscape burned each year on average.

12. The system of claim 1, wherein said lignin and cellulose content of the aboveground plant tissue is measured by sequential digestion of tissue material in a predetermined plot with said predetermined location.

13. A method of certifying the appropriate number of carbon credits associated with a carbon offset project, comprising the steps of:
defining an area of grassland to be used as a carbon credit offset;
establishing a baseline measurement of the soil organic carbon;
measuring the change in sequestered carbon over time; and
obtaining a number of carbon credit corresponding to the change in sequestered carbon over time.

14. The method of claim 13, wherein the step of measuring the change in sequestered carbon over time comprises the steps of:
calculating a maximum aboveground net primary productivity based on an annual rainfall amount;
determining a leaf area index based on a proportion of leaves and a grazing intensity;
calculating an estimated aboveground net primary productivity based on said leaf area index and said maximum aboveground net primary productivity calculating an estimated belowground net primary production;
determining a plant derived soil organic carbon from a fire frequency, a measurement of the lignin and cellulose content of the aboveground plant tissue, and said estimated aboveground net primary productivity;
calculating an amount of dung derived soil organic carbon based on said measurement of the lignin and cellulose content of the aboveground plant tissue, said grazing intensity, and said aboveground net primary productivity;
calculating a carbon loss associated with maintenance respiration; and
determining a change in sequestered carbon by adding said plant derived soil organic carbon to said dung derived soil organic carbon and subtracting said carbon loss associated with maintenance respiration.

15. The method of claim 14, wherein the maximum aboveground net primary productivity based on annual rainfall is weighted by a water holding capacity calculated from a sand content in the predetermined area.

16. The method of claim 14, wherein said grazing intensity comprises the difference in an aboveground biomass under ungrazed conditions and an aboveground biomass under grazed conditions.

17. The method of claim 14, wherein said fire frequency comprises a measurement of the number of fires in said predetermined location every ten years.

18. The method of claim 14, wherein said fire frequency comprises a measurement of the fraction of landscape burned each year on average.

19. The method of claim 14, wherein said lignin and cellulose content of the aboveground plant tissue is measured by sequential digestion of tissue material in a predetermined plot with said predetermined location.
